(19) United States
(12) Patent Application Publication
(10) Pub. No.: US 2015/0361010 A1
(43) Pub. Date: Dec. 17, 2015

(54) APPARATUS AND PROCESS FOR THE CONVERSION OF METHANE INTO ACETYLENE

(71) Applicant: UOP LLC, Des Plaines, IL (US)

(72) Inventors: Laura E. Leonard, Western Springs, IL (US); Rajeswar Gattupalli, Buffalo Grove, IL (US); Peter Shafe, Guildford (GB); Aziz Sattar, West Chicago, IL (US); Richard S. Hatami, Inverness, IL (US)

(21) Appl. No.: 14/301,908

(22) Filed: Jun. 11, 2014

(51) Int. Cl. C07C 2/76; B01J 19/26; B01J 19/10

(52) U.S. Cl. CPC C07C 2/76; B01J 19/10; B01J 19/26; B01J 2219/00162; B01J 2219/0875; B01J 2219/00123; B01J 2219/185

(57) ABSTRACT

A process and apparatus for the pyrolysis of methane into acetylene. A heat exchanger is disposed downstream of a supersonic reactor and is used to recover heat from the quenched effluent. Effluent may flow on a shell side of the heat exchanger and cooling fluid may flow on a tube side. Additionally, a separator is disposed downstream of the heat exchanger so that the effluent is capable of freely draining into the separator. The heat exchanger, separator, or both may be disposed at an angle between 20° to 90° from the horizon so that the fluid is capable of freely draining into the separator. The separator includes an outlet gas valve that may be used to control the pressure within the reactor.

### FIELD OF THE INVENTION

This invention relates to an apparatus and process for converting methane to acetylene with a supersonic reactor.

### BACKGROUND OF THE INVENTION

Light olefin materials, including ethylene and propylene, represent a large portion of the worldwide demand in the petrochemical industry. Light olefins are used in the production of numerous chemical products via polymerization, oligomerization, alkylation and other well-known chemical reactions. These light olefins are essential building blocks for the modern petrochemical and chemical industries. Producing large quantities of light olefin material in an economical manner, therefore, is a focus in the petrochemical industry. The main source for these materials in present day refining is the steam cracking of petroleum feeds.

The cracking of hydrocarbons brought about by heating a feedstock material in a furnace has long been used to produce useful products, including for example, olefin products. For example, ethylene, which is among the more important products in the chemical industry, can be produced by the pyrolysis of feedstocks ranging from light paraffins, such as ethane and propane, to heavier fractions such as naphtha. Typically, the lighter feedstocks produce higher ethylene yields (50-55% for ethane compared to 25-30% for naphtha); however, the cost of the feedstock is more likely to determine which is used. Historically, naphtha cracking has provided the largest source of ethylene, followed by ethane and propane pyrolysis, cracking, or dehydrogenation. Due to the large demand for ethylene and other light olefinic materials, however, the cost of these traditional feeds has steadily increased. In addition, the availability of low cost natural gas has driven interest in the conversion of natural gas to chemicals, such as light olefins.

Energy consumption is another cost factor impacting the pyrolytic production of chemical products from various feedstocks. Over the past several decades, there have been significant improvements in the efficiency of the pyrolysis process that have reduced the costs of production. In a typical or conventional pyrolysis plant, a feedstock passes through a plurality of heat exchanger tubes where it is heated externally to a pyrolysis temperature by the combustion products of fuel oil or natural gas and air. One of the more important steps taken to minimize production costs has been the reduction of the residence time for a feedstock in the heat exchanger tubes of a pyrolysis furnace. Reduction of the residence time increases the yield of the desired product while reducing the production of heavier by-products that tend to foul the pyrolysis tube walls. However, there is little room left to improve the residence times or overall energy consumption in traditional pyrolysis processes.

Recently, attempts have been made to use pyrolysis to convert natural gas to ethylene. U.S. Pat. No. 7,183,451 discloses heating natural gas to a temperature at which a fraction is converted to hydrogen and a hydrocarbon product such as acetylene or ethylene. The product stream is then quenched to stop further reaction and subsequently reacted in the presence of a catalyst to form liquids to be transported. The liquids ultimately produced include naphtha, gasoline, or diesel. While this method may be effective for converting a portion of natural gas to acetylene or ethylene, it is estimated that this approach will provide only about a 40% yield of acetylene from a methane feed stream. While it has been identified that higher temperatures in conjunction with short residence times can increase the yield, technical limitations prevent further improvement to this process in this regard. Furthermore, this reference fails to disclose indirect heat recovery from the pyrolysis reactor.

One proposed alternative to the previous methods of producing olefins that has not gained much commercial traction includes passing a hydrocarbon feedstock into a supersonic reactor and accelerating it to supersonic speed to provide kinetic energy that can be transformed into heat to enable an endothermic pyrolysis reaction to occur. Variations of this process are set out in U.S. Pat. Nos. 4,136,015 and 4,724,272, and Russian Patent No. SU 392723A. These processes include combusting a feedstock or carrier fluid in an oxygen-rich environment to increase the temperature of the feed and accelerate the feed to supersonic speeds. A shock wave is created within the reactor to initiate pyrolysis or cracking of the feed. However, none of these references are believed to disclose the recovery of heat from the reactor effluent.

U.S. Pat. Nos. 5,219,530 and 5,300,216 have suggested a similar process that utilizes a shockwave reactor to provide kinetic energy for initiating pyrolysis of natural gas to produce acetylene. More particularly, this process includes passing steam through a heater section to become superheated and accelerated to a nearly supersonic speed. The heated fluid is conveyed to a nozzle which acts to expand the carrier fluid to a supersonic speed and lower temperature. An ethane feedstock is passed through a compressor and heater and injected by nozzles to mix with the supersonic carrier fluid to turbulently mix together at a speed of about Mach 2.8 and a temperature of about 427° C. The temperature in the mixing section remains low enough to restrict premature pyrolysis. The shockwave reactor includes a pyrolysis section with a gradually increasing cross-sectional area where a standing shock wave is formed by back pressure in the reactor due to flow restriction at the outlet. The shockwave rapidly decreases the speed of the fluid, correspondingly rapidly increasing the temperature of the mixture by converting the kinetic energy into heat. This immediately initiates pyrolysis of the ethane feedstock to convert it to other products. A quench heat exchanger then receives the pyrolized mixture to quench the pyrolysis reaction.

Prior attempts to convert light paraffin or alkane feed streams, including ethane and propane feed streams, to other hydrocarbons using supersonic flow reactors have shown promise in providing higher yields of desired products from a particular feed stream than other more traditional pyrolysis systems. Specifically, the ability of these types of processes to provide very high reaction temperatures with very short associated residence times offers significant improvement over traditional pyrolysis processes. It has more recently been realized that these processes may also be able to convert methane to acetylene and other useful hydrocarbons, whereas more traditional pyrolysis processes were incapable or inefficient for such conversions.

One potential issue associated with using a supersonic flow reactor for light alkane pyrolysis, and more specifically the pyrolysis of methane feeds to form acetylene and other useful products therefrom, is the very large amount of heat must be produced in the supersonic reactor to provide the heat of reaction for the endothermic pyrolysis reactions. In order to generate a large amount of heat and flow rate of the carrier fluid, a large amount of fuel is consumed. Further, at least a portion of the heat must be removed from the process stream after pyrolysis occurs in order to halt the reaction when the desired products have been produced in so that the reactor effluent and other streams may be sent downstream of the supersonic reactor. Moreover, additional heat may be required to preheat a fuel stream or a feed stream.

U.S. Pat Pub. No. 2014/0056767 discloses a supersonic reactor that uses a combustion gas which is provided with a supersonic velocity for the pyrolysis of methane. A quench fluid is injected to stop the pyrolysis reactions. The reactor is free draining or vertically oriented; however, the reference does not disclose using a heat exchange downstream of the reactor. U.S. Pat. Pub. No. 2014/0056766 discloses a similar supersonic reactor that uses a combustion gas which is provided with a supersonic velocity for the pyrolysis of methane. A heat exchanger is used downstream of the reactor. While this reference discloses several general schemes for recovering heat into a heat exchange fluid such as hot oil or to produce high pressure steam that are applicable to the heat exchange fluid discussed herein, it would be desirable, to reduce the amount of fuel and/or energy consumed by such a supersonic reactor and to improve the overall energy efficiency thereof.

### SUMMARY OF THE INVENTION

The present invention provides an apparatus for producing acetylene from a light hydrocarbon stream and a method for recovery of heat from such an apparatus.

In one aspect the present invention provides an apparatus for producing acetylene from a gaseous feed stream comprising light hydrocarbons. The apparatus includes a supersonic reactor configured to receive light hydrocarbons and heat the light hydrocarbons to a pyrolysis temperature to produce a reactor effluent, the supersonic reactor includes a combustion zone capable of combusting a fuel, a pyrolysis zone capable of pyrolyzing light hydrocarbons, a nozzle between the combustion zone and the pyrolysis zone, and, a quench zone configured to receive quench fluid injected into the supersonic reactor to stop the pyrolysis of the light hydrocarbons. The apparatus further includes a separation zone disposed downstream of the quench zone, wherein the reactor effluent is capable of freely draining into the separation zone and separating into a gas phase containing the effluent and a liquid phase containing the quench fluid, and, a heat exchanger disposed between the supersonic reactor and the separation zone.

In some embodiments of the present invention, the supersonic reactor may be vertically orientated.

In at least one embodiment of the present invention, an inlet of the heat exchanger comprises at least a portion of the quench zone of the supersonic reactor.

In various embodiments of the present invention, the heat exchanger comprises a plurality of tubes inside of a shell. It is contemplated that a heat exchange fluid flows on a tube side of the heat exchanger. It is further contemplated that the reactor effluent flows on a shell side of the heat exchanger. The tubes of the heat exchanger may be disposed parallel to a direction of flow through the shell, perpendicular to a direction of flow through the shell, at an angle relative to a direction of flow through the shell, or a combination thereof.

In some embodiments, the separation zone further comprises a pressure control device, such as a valve on an outlet for the gas phase. The separation zone may further include an outlet for removing the liquid phase, which may be configured to supply the liquid phase to the quench zone for use as quench fluid.

In at least one embodiment of the present invention, the heat exchanger further comprises at least one body with an inner cavity and at least one tube or a plurality of tubes extending within the body. Each tube from the plurality of tubes comprises an inlet wherein at least one inlet is disposed in the quench zone. It is contemplated that the tubes are configured to receive reactor effluent. It is further contemplated that cooling fluid flows within the inner cavity of the body of the heat exchanger.

In various embodiments of the present invention, the heat exchanger comprises a plurality of tubes extend within at least one body.

In another aspect of the present invention, the present invention provides a process for producing acetylene from light hydrocarbons in a supersonic reactor. The process includes: injecting light hydrocarbons into a supersonic reactor; heating light hydrocarbons to produce an effluent from a pyrolysis zone; quenching a pyrolysis of light hydrocarbons with a quench fluid to provide a reactor effluent stream comprising effluent and quench fluid; recovering heat from the reactor effluent stream; and, separating the reactor effluent stream in a separation zone into a gas phase comprising the effluent and a liquid phase comprising the quench fluid. The separation zone is disposed so that the reactor effluent stream freely flows into the separation zone from the supersonic reactor.

In some embodiments of the present invention, the heat is recovered in a heat exchanger. The heat exchanger may be disposed between the supersonic reactor and the separation zone. Thus, it is contemplated that the heat exchanger is disposed below the supersonic reactor. The separator may be disposed below the heat exchanger.

The heat exchanger may comprise: a shell with at least one open inner cavity; and, at least one plurality of tubes extending within the at least one open inner cavity. The tubes of the heat exchanger may be disposed parallel, perpendicular, or at an angle to a direction of flow for the reactor effluent stream through the shell. In some embodiments of the present invention, the process also includes reducing a residence time of light hydrocarbon in the supersonic reactor by flowing reactor effluent stream on a tube side of the heat exchanger.

In another embodiment of the present invention, the process includes reducing a pressure drop of light hydrocarbon in the supersonic reactor by flowing reactor effluent on a shell side of the heat exchanger.

In at least one embodiment of the process, the process further includes adjusting the pressure in the supersonic reactor by controlling a flow of gas out of the separation zone.

These and other aspects and embodiments relating to the present invention should be apparent to those of ordinary skill in the art from the following detailed description of the invention.

### DETAILED DESCRIPTION OF THE DRAWINGS

In the drawings:

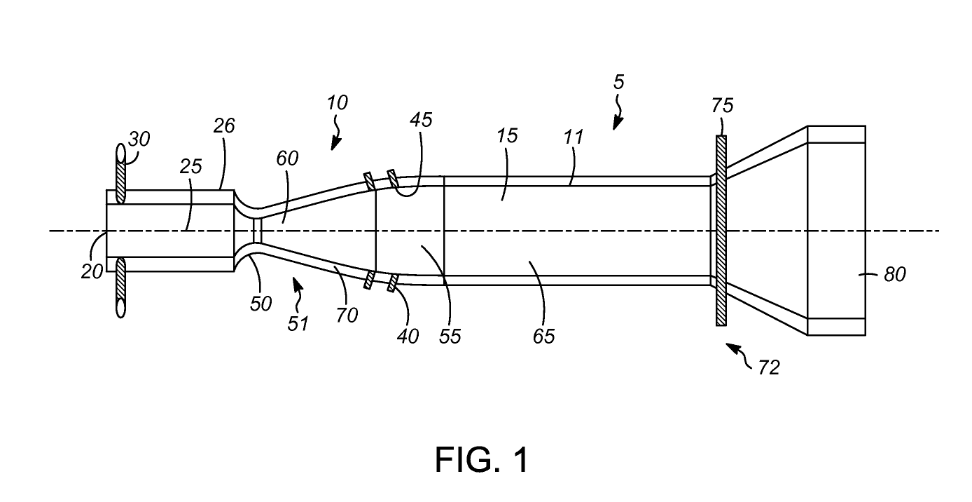

FIG. 1 is a side cutaway view of a supersonic reactor used in accordance with various embodiments of the present invention;

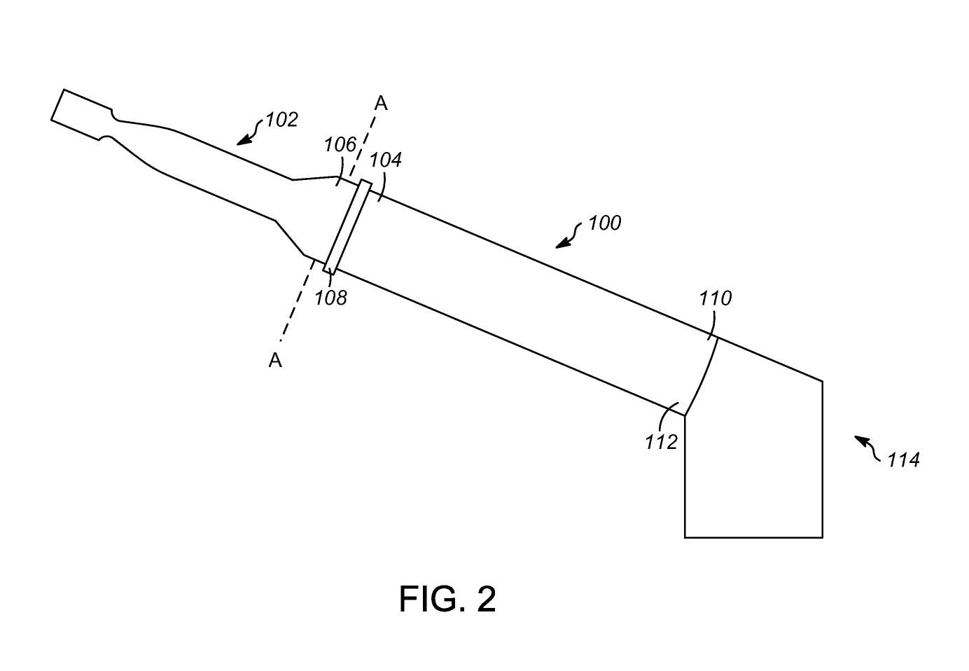

FIG. 2 is a side view of an apparatus according to one or more embodiments of the present invention;

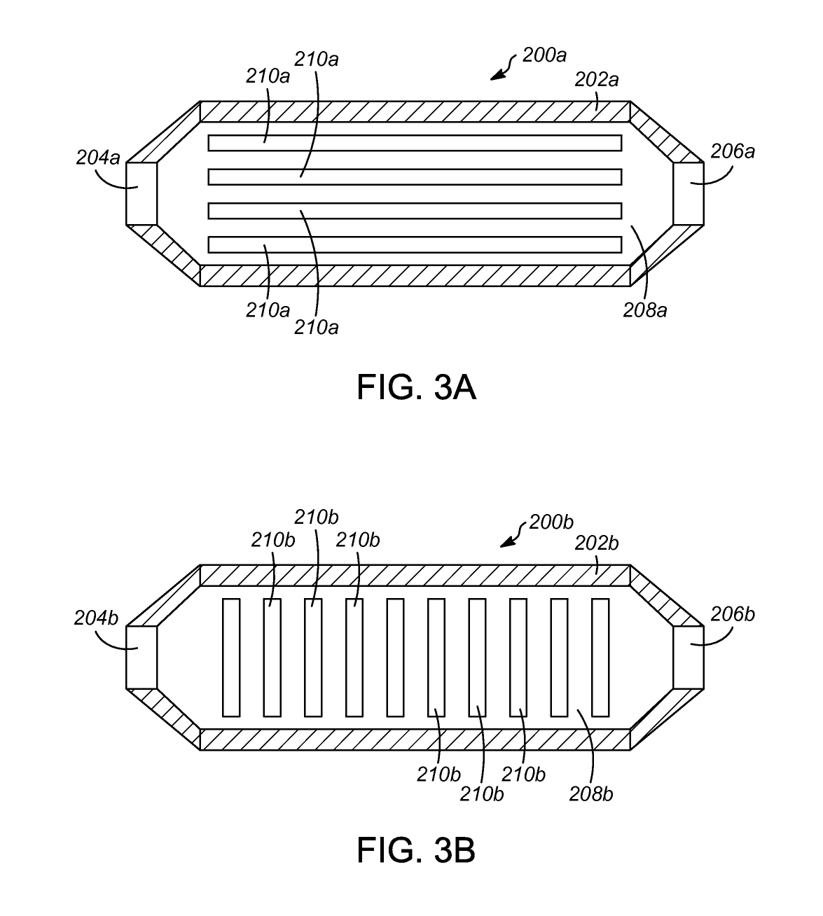

FIG. 3A is a side cutaway view of a heat exchanger used in one or more embodiments of the present invention;

FIG. 3B is a side cutaway view of another heat exchanger used in one or more embodiments of the present invention;

FIG. 3C is a side cutaway view of yet another heat exchanger used in one or more embodiments of the present invention;

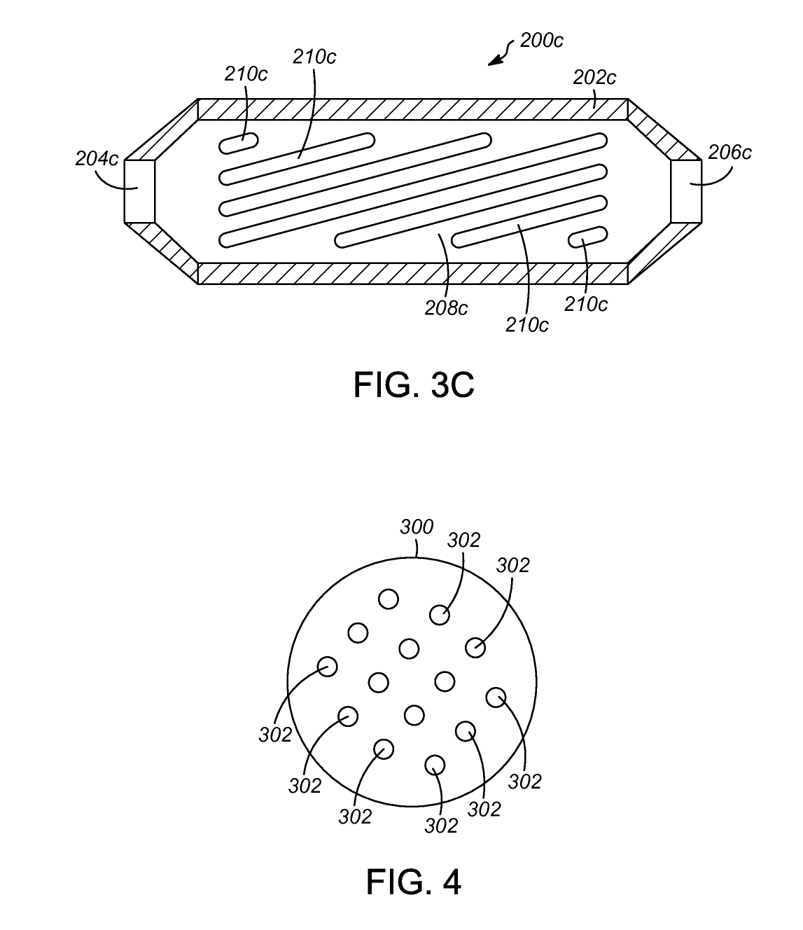

FIG. 4 is a top view of a cutaway of the apparatus of FIG. 2 taken along line A-A; and,

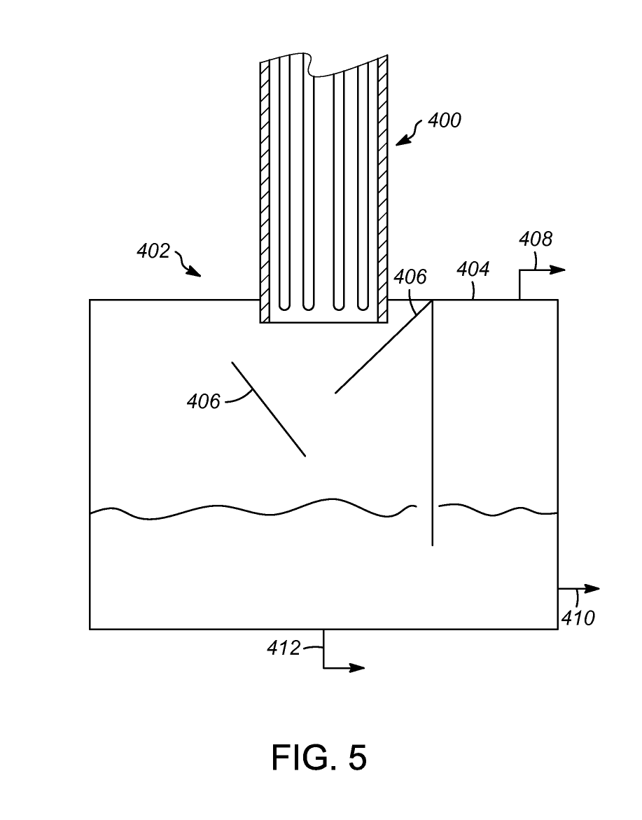

FIG. 5 is aside cutaway view of a separator used in accordance with various embodiments of the present invention.

### DETAILED DESCRIPTION OF THE INVENTION

An apparatus and method have been developed in which for producing acetylene from a feed stream containing light hydrocarbons. By “light hydrocarbons,” it is meant that the feed stream comprises methane to C₄ hydrocarbons (i.e., hydrocarbons with four carbon atoms), and may also comprise trace or small amounts of C₅+ hydrocarbons (i.e., hydrocarbons with five or more carbon atoms). The feed stream may be provided from a remote location or at the location or locations of the systems and methods described herein. For example, while the feed stream source may be located at the same refinery or processing plant where the processes and systems are carried out, such as from production from another on-site hydrocarbon conversion process or a local natural gas field, the feed stream may be provided from a remote source via pipelines or other transportation methods. For example a feed stream may be provided from a remote hydrocarbon processing plant or refinery or a remote natural gas field, and provided as a feed to the systems and processes described herein. Initial processing of the feed stream may occur at the remote source to remove certain contaminants from the feed stream. Where such initial processing occurs, it may be considered part of the systems and processes described herein, or it may occur upstream of the systems and processes described herein. Thus, the feed stream provided for the systems and processes described herein may have varying levels of contaminants depending on whether initial processing occurs upstream thereof.

In one example, the feed stream has a methane content ranging from about 65 mol-% to about 100 mol-%. In another example, the concentration of methane in the hydrocarbon feed stream ranges from about 80 mol-% to about 100 mol-%. In yet another example, the concentration of methane ranges from about 90 mol-% to about 100 mol-% of the hydrocarbon feed.

In one example, the concentration of ethane in the feed stream ranges from about 0 mol-% to about 35 mol-% and in another example from about 0 mol-% to about 10 mol-%. In one example, the concentration of propane in the methane feed ranges from about 0 mol-% to about 5 mol-% and in another example from about 0 mol-% to about 1 mol-%.

The feed stream may also include heavier hydrocarbons, including aromatics, paraffinic, olefinic, and naphthenic hydrocarbons. These heavier hydrocarbons if present will likely be present at concentrations of between about 0 mol-% and about 100 mol-%. In another example, they may be present at concentrations of between about 0 mol-% and 10 mol-% and may be present at between about 0 mol-% and 2 mol-%.

The apparatus and method for forming acetylene from the light hydrocarbon feed stream described herein utilizes a supersonic flow reactor for pyrolyzing methane in the feed stream to form acetylene. The supersonic flow reactor may include one or more reactors capable of creating a supersonic flow of a carrier fluid and the methane feed stream and expanding the carrier fluid to initiate the pyrolysis reaction. A preferred reactor is depicted in U.S. Pat. Pub. No. 2014/0056766, which is incorporated herein by reference, in its entirety.

As illustrated in FIG. 1, an apparatus according to the present invention includes a supersonic reactor 5. The reactor 5 may include a reactor vessel 10 generally defining a reactor chamber 15. While the reactor 5 is illustrated as a single reactor, it should be understood that it may be formed modularly or as separate vessels. If formed modularly or as separate components, the modules or separate components of the reactor may be joined together permanently or temporarily, or may be separate from one another with fluids contained by other means, such as, for example, differential pressure adjustment between them. A combustion zone or chamber 25 is provided for combusting a fuel to produce a carrier fluid with the desired temperature and flow rate. The reactor 5 may optionally include a carrier fluid inlet 20 for introducing a supplemental carrier fluid into the reactor. One or more fuel injectors 30 are provided for injecting a combustible fuel, for example hydrogen, into the combustion chamber 25. The same or other injectors may be provided for injecting an oxygen source into the combustion chamber 25 to facilitate combustion of the fuel. The fuel and oxygen are combusted to produce a hot carrier fluid stream typically having a temperature of from about 1200° C. to about 3500° C. in one example, between about 2000° C. and about 3500° C. in another example, and between about 2500° C. and about 3200° C. in yet another example. It is also contemplated herein to produce the hot carrier fluid stream by other known methods, including non-combustion methods. According to one example the carrier fluid stream has a pressure of about 1 atm or higher, greater than about 2 atm in another example, and greater than about 4 atm in another example. The hot carrier fluid stream from the combustion zone 25 is passed through a supersonic expander 51 that includes a converging-diverging nozzle 50 to accelerate the flow rate of the carrier fluid to above about Mach 1.0 in one example, between about Mach 1.0 and Mach 4.0 in another example, and between about Mach 1.5 and 3.5 in another example. In this regard, the residence time of the fluid in the reactor portion of the supersonic flow reactor is between about 0.1-100 ms in one example, about 0.1-50 ms in another example, and about 0.1-20 ms in another example. The temperature of the carrier fluid stream through the supersonic expander by one example is between about 1000° C. and about 3500° C., between about 1200° C. and about 2500° C. in another example, and between about 1200° C. and about 2000° C. in another example.

A feed stream inlet 40 is provided for injecting the light hydrocarbon feed stream into the reactor 5 to mix with the carrier fluid. The feed stream inlet 40 may include one or more injectors 45 for injecting the feed stream into the nozzle 50, a mixing zone 55, an expansion zone 60, or a reaction zone or chamber 65. The injector 45 may include a manifold, including for example a plurality of injection ports or nozzles for injecting the feed stream into the reactor 5.

As illustrated in FIG. 1, the reactor 5 may have a separate mixing zone, between for example the supersonic expander 51 and reaction zone 65. Optionally, a supersonic diffuser or a second converging-diverging nozzle may be positioned between mixing zone 55 and reaction zone 65.

In another approach, the mixing zone is integrated into the expansion zone 60 is provided, and mixing may occur in the nozzle 50, expansion zone 60, or reaction zone 65 of the reactor 5. A combination of geometric features such as for example a super-sonic diffuser and manipulation of the pressure at the outlet end of reaction zone 65 may be utilized to produce a rapid reduction in the velocity of the gases flowing therethrough, to convert the kinetic energy of the flowing fluid to thermal energy to further heat the stream to cause pyrolysis of the methane in the feed stream in reaction zone 65 of the reactor 5. A quench fluid is injected into the reactor 5 in a quench zone 72 to stop the pyrolysis reaction from further conversion of the desired acetylene product to other compounds. Any suitable structure may be utilized for the introduction of quench fluid. For example, spray bars 75 may be used to introduce a quenching fluid, for example water or steam, into the quench zone 72.

The reactor effluent exits the reactor via outlet 80 will include a larger concentration of acetylene than the feed stream and a reduced concentration of methane relative to the feed stream. The reactor effluent stream may also be referred to herein as an acetylene stream as it includes an increased concentration of acetylene. The acetylene stream may be an intermediate stream in a process to form another hydrocarbon product or it may be further processed and captured as an acetylene product stream. In one example, the reactor effluent stream has an acetylene concentration prior to the addition of quenching fluid ranging from about 2 mol-% to about 30 mol-%. In another example, the concentration of acetylene ranges from about 5 mol-% to about 25 mol-% and from about 8 mol-% to about 23 mol-% in another example.

The reactor vessel 10 includes a reactor shell 11. It should be noted that the term “reactor shell” refers to the wall or walls forming the reactor vessel, which defines the reactor chamber 15. The reactor shell 11 will typically be an annular structure defining a generally hollow central reactor chamber 15. The reactor shell 11 may include a single layer of material, a single composite structure or multiple shells with one or more shells positioned within one or more other shells. The reactor shell 11 also includes various zones, components, and or modules, as described above and further described below for the different zones, components, and or modules of the supersonic reactor 5. The reactor shell 11 may be formed as a single piece defining all of the various reactor zones and components or it may be modular, with different modules defining the different reactor zones and/or components.

Turning to FIG. 2, at least one heat exchanger 100 is provided downstream of a supersonic reactor 102, for recovering heat from at least a portion of the supersonic reactor 102 effluent stream. The reactor preferably includes all of the features of reactor 5 shown in FIG. 1. The recovered heat may be transferred to one or more other portions of the process stream. The process stream may include any of the process streams described above, or may include other process streams, including, for example, dedicated heat transfer process streams. The dedicated heat transfer process streams may comprise any phase or combination of phases, as further described herein. For example, the heat transfer fluid may comprise a hydrocarbon stream, hot oil to feed a hot oil heat exchange loop, or water or steam to provide process heat or generate power as disclosed in U.S. Pat. Pub. No. 2014/0056766.

An exemplary heat exchanger 200a is shown in FIG. 3A, in which the heat exchanger 200a is a transfer line heat exchanger. Other heat exchangers may be used, include a tube-in-tube design, such as that disclosed in U.S. Pat. No. 8,177,200. A tube-in-tube design, may be configured such that the reactor effluent flows through the inner tube and the heat exchange fluid flows through the outer annulus or shell.

As shown in FIG. 3A, the heat exchanger 200a includes a shell body 202a with an inlet 204a, an outlet 206a and an open inner cavity 208a. A plurality of tubes 210a extend within the open inner cavity 208a. Generally, the reactor effluent from the reactor 102 will flow in direction from the inlet 204a to the outlet 206a. As shown, the tubes 210a may be orientated in a direction that is parallel to the flow of the reactor effluent.

Alternatively, in FIG. 3B, a heat 200b exchanger is shown which also includes 202b with an inlet 204b, an outlet 206b, an open inner cavity 208b, and a plurality of tubes 210b within the open inner cavity 208b. In this embodiment, the tubes 210b are generally perpendicular to the direction of flow of the reactor effluent (from inlet 204b to outlet 206b).

Finally, as shown in FIG. 3C, a heat 200c exchanger is shown which also includes 202c with an inlet 204c, an outlet 206c, an open inner cavity 208c, and a plurality of tubes 210c within the open inner cavity 208c. In this embodiment, the tubes 210c are at an angle (between being perpendicular and being parallel) to the direction of flow of the reactor effluent (from inlet 204c to outlet 206c). Furthermore, the use of the terms “perpendicular” and “parallel” is intended to include configurations that are relatively parallel or perpendicular (i.e., +/−approximately 10°). It is further contemplated that a combination of the various orientations of the tubes may be used.

Returning to FIG. 2, it is contemplated that an inlet 104 of the heat exchanger 100 is disposed in the quench zone 106 of the reactor. The heat exchanger 100 may be connected to the reactor 102 with, for example, a flange 108. It is further contemplated that an optional second quench zone 110 is disposed within the outlet 112 of the heat exchanger 100.

In an embodiment, the reactor effluent flows, for example with respect to FIG. 3B, in the open inner cavity 208b of the shell 202b (or on the shell side) of the heat exchanger 200b. Accordingly, cooling fluid preferably flows in the tubes 210b (or on the tube side) of the heat exchanger 200b. The cooling fluid may comprise water, steam, a hydrocarbon stream or hot oil, or any other process stream requiring heat input. By flowing the reactor effluent and quench fluid from the reactor 102 on the shell side of the heat exchanger 100, a pressure drop associated with the flow of the fluids into the heat exchanger can be minimized.

Alternatively, it is contemplated that the reactor effluent flow on the tube side of the heat exchanger 100. In such an embodiment, it is contemplated that the heat exchange inlet 108 will be disposed within the quench zone 106 of the reactor 102. The inlet 108 of the heat exchanger 100 may include a distribution plate 300. See, FIG. 4. The distribution plate 300 includes a plurality of apertures 302 which act as inlets for the tubes extending through the inner cavity of the heat exchanger. In this embodiment, it may be preferable to enclose each tube in an individual shell or to provide a single shell to enclose multiple tubes as is shown in FIG. 3A where the tubes are parallel to the direction of flow. Cooling fluid will flow outside of the tubes, for example, on the shell side of the heat exchanger or through the outer annulus of a tube-in-tube design. Such a configuration is believed to provide for a more uniform heat recovery, which can reduce the energy consumption of the overall process because the enthalpy contained in the pyrolysis product is recovered as usable heat, for example high quality steam. Such a design may also provide the desired heat recovery with low residence time, for example <100 ms from the inlet of the quench zone to the outlet of the heat exchanger, while also providing acceptable pressure drop, for example <5 psi or <2 psi. The advantage of such a configuration is that the quench zone may act as the distribution zone feeding the tubes of the heat exchanger.

As mentioned above, the heat recovered from the heat exchanger may used in the present process, for example to pre-heat certain process streams. Additionally and alternatively, the heat may be used elsewhere, for example, for the production of electricity. The recovery of the heat is not necessary for the understanding and the practicing of the present invention.

In at least one embodiment of the present invention, the effluent is cooled from 1000 to 1500° C. to 600-900° C. in the quench zone and subsequently cooled from 600-900° C. in the heat exchanger. In at least one embodiment of the present invention, the residence time of the effluent in the quench zone is less than 10 ms and the residence time of the effluent in the heat exchanger is greater than 30 ms or between about 30 ms and about 200 ms or about 30 ms and about 100 ms. In some embodiments the residence time for effluent with a temperature between 1000 to 1500° C. is less than 5 ms, for effluent with a temperature between 650 to 1000° C. is less than 10 ms, and for effluent with a temperature between 200 to 650° C. is less than 40 ms.

Returning to FIG. 2, in various embodiments of the present invention, a separation zone 114 is disposed downstream of the reactor 102. The separation zone 114 is preferably disposed downstream of both the heat exchanger 100 and the reactor 102. In accordance with various embodiments of the present invention the separation zone 114 is disposed so that the reactor effluent is capable of freely draining into the separation zone 114. As used herein, “capable of freely draining” means that the reactor (and any piping or equipment, such as a heat exchanger, separating the reactor and separation zone) is disposed at an angle from at least 20° up to 90° (i.e., vertical) from the horizon.

For example, the reactor may be a relatively vertically orientated reactor (90°), with the heater exchanger disposed below the reactor, and with the separation zone disposed below the heat exchanger By arranging the separation zone so that the reactor effluent is capable of freely draining into the separation zone, any pressure drop associated with the transfer of fluids between the various components or zones can be minimized. Furthermore, excess cooling fluid that may be injected during normal operation or during transient periods such as start-up or shutdown will not accumulate in the reactor.

As shown in FIG. 5, a heat exchanger 400 is disposed above a separation zone 402 which includes at least one separator vessel 404. If there are a plurality of reactors in the reaction zone, each reactor may include a separator vessel, or each reactor may drain into the same separator vessel. The separator vessel 404 may include one or more baffles 406 to direct the flow of fluids (both liquid and gas) and aid in the separation of the gas and liquid phases. In the separator vessel 404, the reactor effluent separates into a gas phase comprising at least acetylene and a liquid phase comprising the quench fluid.

As shown in FIG. 5, the separator vessel 404 may include a gas outlet 408, a liquid outlet 410, and a solid outlet 412. The acetylene and other gaseous components of the effluent may be recovered via the gas outlet 408. The gas outlet 408 may also include a pressure control device such as a control valve which can be used to allow for pressure control within the reactor. For example, adjusting the pressure may allow for the shock zone of the reactor to be adjusted. The liquid outlet of the separator vessel 404 can be used to recover excess quench fluid. This may be especially beneficial during times when excess quench fluid is injected into the reactor (during start up for example). Since all of the quench fluid may not evaporate during certain times, the separator is configured to receive same. Finally, any soot or other solid material may be recovered from the separator vessel 404 via the solid outlet 412.

An apparatus according to one or more embodiments herein provides for efficient heat recovery from a supersonic reactor. Efficiently recovering heat from the reaction effluent, is believed to allow for the reactor to be run more efficiently. In various embodiments, the efficient recovery can be performed while utilizing a heat exchanger without excessively increasing residence time in the reactor for the effluent, or without creating too large of a pressure drop.

It should be appreciated and understood by those of ordinary skill in the art that various other components such as valves, pumps, filters, coolers, etc. were not shown in the drawings as it is believed that the specifics of same are well within the knowledge of those of ordinary skill in the art and a description of same is not necessary for practicing or understating the embodiments of the present invention.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. An apparatus for producing acetylene from a gaseous feed stream comprising light hydrocarbons, the apparatus comprising:
a supersonic reactor configured to receive light hydrocarbons and heat the light hydrocarbons to a pyrolysis temperature to produce a reactor effluent, the supersonic reactor including: a combustion zone capable of combusting a fuel; a pyrolysis zone capable of pyrolyzing light hydrocarbons; a nozzle between the combustion zone and the pyrolysis zone; and, a quench zone configured to receive quench fluid injected into the supersonic reactor to stop the pyrolysis of the light hydrocarbons;
a separation zone disposed downstream of the quench zone, wherein the reactor effluent is capable of freely draining into the separation zone and separating into a gas phase containing the effluent and a liquid phase containing the quench fluid; and,
a heat exchanger disposed between the supersonic reactor and the separation zone.

2. The apparatus of claim 1 wherein the supersonic reactor is vertically orientated.

3. The apparatus of claim 1 wherein an inlet of the heat exchanger comprises at least a portion of the quench zone of the supersonic reactor.

4. The apparatus of claim 3 wherein the heat exchanger comprises a plurality of tubes inside of a shell.

5. The apparatus of claim 4 wherein a heat exchange fluid flows on a tube side of the heat exchanger.

6. The apparatus of claim 4 wherein the reactor effluent flows on a shell side of the heat exchanger.

7. The apparatus of claim 4 wherein the tubes of the heat exchanger are disposed parallel, perpendicular, at an angle or a combination thereof to a direction of flow through the shell.

8. The apparatus of claim 1 wherein the heat exchanger further comprises at least one body having an inner cavity and at least one tube extending within the body.

9. The apparatus of the claim 8 wherein each tube from the plurality of tubes comprises an inlet wherein at least one inlet is disposed in the quench zone.

10. The apparatus of claim 8 wherein the tubes are configured to receive reactor effluent.

11. The apparatus of claim 10 wherein cooling fluid flows within the inner cavity of the body of the heat exchanger.

12. The apparatus of claim 8, wherein the heat exchanger comprises a plurality of tubes extend within at least one body.

13. The apparatus of claim 1 wherein the separation zone further comprises a pressure control device.

14. A process for producing acetylene from light hydrocarbons in a supersonic reactor, the process comprising:
injecting light hydrocarbons into a supersonic reactor;
heating light hydrocarbons to produce an effluent from a pyrolysis zone;
quenching a pyrolysis of light hydrocarbons with a quench fluid to provide a reactor effluent stream comprising effluent and quench fluid;
recovering heat from the reactor effluent stream; and,
separating the reactor effluent stream in a separation zone into a gas phase comprising the effluent and a liquid phase comprising the quench fluid; and,
wherein the separation zone is disposed so that the reactor effluent stream freely flows into the separation zone from the supersonic reactor.

15. The process of claim 14 wherein the heat is recovered in a heat exchanger and wherein the heat exchanger is disposed between the supersonic reactor and the separation zone.

16. The process of claim 15 wherein the heat exchanger comprises: a shell with at least one open inner cavity and at least one tube or a plurality of tubes extending within the at least one open inner cavity.

17. The process of claim 16 wherein the tubes of the heat exchanger are disposed parallel, perpendicular, at an angle or a combination thereof to a direction of flow for the reactor effluent stream through the shell.

18. The process of claim 16 further comprising:
reducing a residence time of light hydrocarbon in the supersonic reactor by flowing reactor effluent stream on a tube side of the heat exchanger.

19. The process of claim 16 further comprising:
reducing a pressure drop of light hydrocarbon in the supersonic reactor by flowing reactor effluent stream on a shell side of the heat exchanger.

20. The process of claim 14 further comprising:
adjusting the pressure in the supersonic reactor by controlling a flow of gas out of the separation zone.
